SEQUENCE LISTING (4 sequences)

SEQ ID NO: 1 - protein, 53 aa, Homo Sapiens
NSDSECPLSHDGYCLHDGVCMYIEALDKYACNCVVGYIGERCQYRDLKWWELR

SEQ ID NO: 2 - protein, 53 aa, Pig
NSYSECPPSHDGYCLHGGVCMYIEAVDSYACNCVFGYVGERCQHRDLKWWELR

SEQ ID NO: 3 - protein, 52 aa, Cat
NSYQECPPSYDGYCLYNGVCMYIEAVDRYACNCVFGYVGERCQHRDLKWELR

SEQ ID NO: 4 - protein, 52 aa, Dog
NGYRECPSSYDGYCLYNGVCMYIEAVDRYACNCVFGYVGERCQHRDLKWELR

(19) United States
(12) Patent Application Publication
(10) Pub. No.: US 2008/0233614 A1
(43) Pub. Date: Sep. 25, 2008

(54) PRODUCTION OF RECOMBINANT COLLAGENASES COLG AND COLH IN ESCHERICHIA COLI

(76) Inventors: Rocky M. Cranenburgh, Staffordshire (GB); Gregory L. Sabatino, Chester Springs, PA (US); Benjamin J. Del Tito, Doylestown, PA (US)

Correspondence Address: ELMORE PATENT LAW GROUP, PC, 515 Groton Road, Unit 1R, Westford, MA, 01886, US

(21) Appl. No.: 12/028,849

(22) Filed: Feb. 11, 2008

Related U.S. Application Data
(60) Provisional application No. 60/889,666, filed on Feb. 13, 2007.

(51) Int. Cl. C12P 21/04; C12N 15/11; C12N 15/00; C12N 1/20

(52) U.S. Cl. 435/69.1; 536/23.2; 435/320.1; 435/252.33

(57) ABSTRACT

The present invention provides codon-optimized genes designed to help maximize heterologous protein expression level. The present invention provides codon-optimized recombinant colG and colH collagenase sequences.

### RELATED APPLICATION

This application claims the benefit of U.S. Provisional Application No. 60/889,666, filed on Feb. 13, 2007. The entire teachings of the above application are incorporated herein by reference.

### BACKGROUND OF THE INVENTION

In most organisms, synonymous codons are not used equally and in many unicellular organisms, like Escherichia coli, the preferential use of some codons varies from gene to gene and the strength of the preference—the codon bias—increases in genes at high expression level (Gouy, M. and Gautier, C. (1982) Nucleic Acids Res., 10, 7055-7074). This suggests that there is a positive selection on codons that are translated more efficiently, either faster or more accurately. It has been shown that the strength of the codon bias to some extent also depends on gene length (Eyre-Walker, A. (1996) Mol. Biol. Evol., 13, 864-872) and on the context of bases surrounding each codon (Yarus, M. and Folley, L. S. (1985) J. Mol. Biol., 182, 529-540; Gouy, M. (1987) Mol. Biol. Evol., 4, 426-444; Berg, O. G. and Silva, P. J. N. (1997) Nucleic Acids Res., 25, 1397-1404).

Collagenase is an enzyme that has the specific ability to digest collagen and collagenase injections have been proposed for the treatment of diseases such as Duptyren's disease and Peyronie's disease. Both diseases are associated with collagen plaques or cords. Wegman, Thomas L., U.S. Pat. No. 5,589,171, Dec. 31, 1996, U.S. Pat. No. 6,086,872, Jul. 11, 2000 and U.S. Pat. No. 6,022,539, Feb. 8, 2000, which are incorporated herein by reference. The collagenase enzyme has been used to treat a variety of collagen-mediated diseases and collagenase for use in therapy has been obtained from a variety of sources including mammalian (e.g. human), crustacean (e.g. crab, shrimp), fungal, and bacterial (e.g. from the fermentation of Clostridium, Streptomyces, Pseudomonas, or Vibrio). One common source of crude collagenase is from a bacterial fermentation process, specifically the fermentation of C. histolyticum (C. his) which must then be purified. One drawback of the fermentation process from C. his is that it yields uncertain ratios of the various collagenases such as collagenase I and collagenase II. Further, the culture has historically required the use of meat products.

Various ratios of collagenase I to collagenase II in a therapeutic collagenase preparation have different biological effects. Therefore, a therapeutic collagenase preparation in which the ratio of collagenase I to collagenase II in the preparation can be easily and efficiently determined and controlled to obtain superior, and consistent enzyme activity and therapeutic effect, would be desirable.

Because preferential usage of different synonymous codons by E. coli (codon bias) can negatively affect expression levels of recombinant proteins, and because this reduced expression directly affects the yield of pure protein product, there remains a need for methods to neutralize or minimize the effects of codon bias.

### SUMMARY OF THE INVENTION

According to the present invention, codon-optimized colG and colH genes were designed to help maximize the heterologous protein expression level. The present invention provides codon-optimized recombinant collagenase sequences.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a map of each of the four collagenase expression plasmids.

FIG. 2 is a gel of the restriction analysis of the four expression plasmids.

2A

| pARA-ColG | pARA-ColH |
|---|---|
| Lane 1: 1 kb Plus Ladder | Lane 1: 1 kb Plus Ladder |
| Lane 2: Uncut | Lane 2: Uncut |
| Lane 3: NdeI (6.7 kb) | Lane 3: NdeI (6.6 kb) |
| Lane 4: SalI, EcoRI (1.2 kb, 5.5 kb) | Lane 4: NdeI, SacII (2.3 kb, 4.3 kb) |
| Lane 5: NdeI, SalI (3.1 kb, 3.6 kb) | Lane 5: NdeI, SalI (3.0 kb, 3.6 kb) |
| Lane 6: HindIII (0.8 kb, 1.2 kb, 4.7 kb) | Lane 6: SfiI (1.0 kb, 2.2 kb, 3.3 kb) |
| Lane 7: 1 kb Plus Ladder | Lane 7: 1 kb Plus Ladder |

2B

| pLPR-ColG | pLPR-ColH |
|---|---|
| Lane 1: 1 kb Plus Ladder | Lane 1: 1 kb Plus Ladder |
| Lane 2: Uncut | Lane 2: Uncut |
| Lane 3: NdeI (7.4 kb) | Lane 3: NdeI (7.4 kb) |
| Lane 4: NdeI, SalI (3.0 kb, 4.4 kb) | Lane 4: NdeI, SalI (3.0 kb, 4.4 kb) |
| Lane 5: NdeI, EcoRI (1.2 kb, 1.9 kb, 4.3 kb) | Lane 5: BglII (0.8 kb, 2.6 kb, 4.0 kb) |
| Lane 6: 1 kb Plus Ladder | Lane 6: NdeI, SacII (2.3 kb, 5.0 kb) |
| | Lane 7: 1 kb Plus Ladder |

FIG. 3 is a gel illustrating the stability of plasmids pARA-ColG, pARA-ColH, pLPR-ColG and pLPR-ColH.

Lanes 1, 16, 17 and 24: Stratagene 1 kb DNA size marker

Lane 2: pARA-colG

Lanes 3 to 8: pARA-colG stability study, day 0 to day 5

Lane 9: pARA-colH

Lanes 5 to 15: pARA-colH stability study, day 0 to day 5

Lane 18: pLPR-colG

Lanes 19 to 24: pLPR-colG stability study, day 0 to day 5

Lane 25: pLPR-colH

Lanes 26 to 31: pLPR-colH stability study, day 0 to day 5

FIG. 4 is an SDS-PAGE analysis of protein expression from pARA-ColG, pARA-ColH, pLPRColG and pLPR-ColH in TOP 10 cells.

ColG
Lane 1: Mark 12 size marker
Lane 2: 0.5 μg ColG reference
Lane 3: t0, (pARA-ColG)
Lane 4: t2, (pARA-ColG)
Lane 5: t4, (pARA-ColG)
Lane 6: t0, (pLPR-ColG)
Lane 7: t1, (pLPR-ColG)
Lane 8: t3, (pLPR-ColG)
ColH
Lane 9: 0.5 μg ColH reference
Lane 10: t0, (pARA-ColH)
Lane 11: t2, (pARA-ColH)
Lane 12: t4, (pARA-ColH)
Lane 13: t0, (pLPR-ColH)
Lane 14: t1, (pLPR-ColH)
Lane 15: t3, (pLPR-ColH)

FIG. 5 is a western blot of ColG expression from the arabinose and lambda promoters.

Lane 1: 50 ng ColG reference; Prestained SeeBlue

Plus2 Molecular Weight Marker (Invitrogen)

Lane 2: t0, (pARA-ColG)

Lane 3: t2, (pARA-ColG)

Lane 4: t4, (pARA-ColG)

Lane 5: t0, (pLPR-ColG)

Lane 6: t1, (pLPR-ColG)

Lane 7: t3, (pLPR-ColG)

FIG. 6 is a western blot of ColH expression from the arabinose and lambda promoters.

Lane 1: 50 ng ColH reference; Prestained SeeBlue

Plus2 Molecular Weight Marker (Invitrogen)

Lane 2: t0, (pARA-ColH)

Lane 3: t2, (pARA-ColH)

Lane 4: t4, (pARA-ColH)

Lane 5: t0, (pLPR-ColH)

Lane 6: t1, (pLPR-ColH)

Lane 7: t3, (pLPR-ColH)

FIG. 7 is an SDS-PAGE analysis of the soluble and insoluble fractions of ColG and ColH in the arabinose system at 37° C.

Lane 1: Mark12 size marker Invitrogen)

Lane 2: t0, (pARA-ColG), soluble

Lane 3: t0, (pARA-ColG), insoluble

Lane 4: t2, (pARA-ColG), soluble

Lane 5: t2, (pARA-ColG), insoluble

Lane 6: t0, (pARA-ColH), soluble

Lane 7: t0, (pARA-ColH), insoluble

Lane 8: t2, (pARA-ColH), soluble

Lane 9: t2, (pARA-ColH), insolube

FIG. 8 is an SDS-PAGE analysis of ColG expression in the arabinose system at 30° C.

Lanes 1 and 9: Mark12 size marker (Invitrogen)

Lane 2: 0.5 μg ColG reference

Lane 3: t0, (pARA-ColG), whole cell

Lane 4: t0, (pARA-ColG), soluble

Lane 5: t0, (pARA-ColG), insoluble

Lane 6: t3, (pARA-ColG), whole cell

Lane 7: t3, (pARA-ColG), soluble

Lane 8: t3, (pARA-ColG), insoluble

FIG. 9 is an SDS-PAGE analysis of ColH expression in the arabinose system at 30° C.

Lanes 1 and 9: Mark12 size marker (Invitrogen)

Lane 2: 0.5 μg ColH reference

Lane 3: t0, (pARA-ColH), whole cell

Lane 4: t0, (pARA-ColH), soluble

Lane 5: t0, (pARA-ColH), insoluble

Lane 6: t3, (pARA-ColH), whole cell

Lane 7: t3, (pARA-ColH), soluble

Lane 8: t3, (pARA-ColH), insoluble

FIG. 10 is a western blot showing expression of ColG from the arabinose system at 30° C. and 37° C.

Lane 1: Magic marker (Invitrogen)

Lane 2: 50 ng ColG reference protein

Lane 3: t0, (pARA-ColG), soluble at 30° C.

Lane 4: t0, (pARA-ColG), insoluble at 30° C.

Lane 5: t3, (pARA-ColG), soluble at 30° C.

Lane 6: t3, (pARA-ColG), insoluble at 30° C.

Lane 7: t0, (pARA-ColG), soluble at 37° C.

Lane 8: t0, (pARA-ColG), insoluble at 37° C.

Lane 9: t2, (pARA-ColG), soluble at 37° C.

Lane 10: t2, (pARA-ColG), insoluble at 37° C.

FIG. 11 is a gel showing the soluble and insoluble fractions of ColG and ColH in the lambda system by SDS-PAGE.

Lane 1: Mark12 size marker (Invitrogen)

Lane 2: t0, (pLPR-ColG), soluble

Lane 3: t0, (pLPR-ColG), insoluble

Lane 4: t3, (pLPR-ColG), soluble

Lane 5: t3, (pLPR-ColG), insoluble

Lane 6: t0, (pLPR-ColH), soluble

Lane 7: t0, (pLPR-ColH), insoluble

Lane 8: t3, (pLPR-ColH), soluble

Lane 9: t3, (pLPR-ColH), insoluble

FIG. 12 is a western blot of ColG and ColH expression from the lambda system.

Lanes 1 and 6: Size marker.

Lane 2: t0, pLPR-ColG, soluble

Lane 3: t0, pLPR-ColG, insoluble

Lane 4: t3, pLPR-ColG, soluble

Lane 5: t3, pLPR-ColG, insoluble

Lane 7: t0, pLPR-ColH, soluble

Lane 8: t0, pLPR-ColH, insoluble

Lane 9: t3, pLPR-ColH, soluble

Lane 10: t3, pLPR-ColH, insoluble

### DETAILED DESCRIPTION OF THE INVENTION

A description of preferred embodiments of the invention follows.

According to the present invention, collagenase genes ColH and ColG were codon-optimized to avoid the potential problem of reduced yields when expressing heterologous proteins in E. coli. The corresponding amino acid sequences of the synthesized genes were identical to sequences published in Genbank by Matsushita et al. (1999) and Yoshihara et al. (1994) for Clostridium histolyticum strain JCM 1403 (ATCC 19401). The codon-optimized gene sequence provide an identical amino acid sequence to the mature protein with the signal peptide cleaved, with the exception that the N-terminal methionine is unlikely to be cleaved due to the nature of the second amino acid in the sequence (which determines cleavage efficiency) and the mechanism of production as inclusion bodies. N-terminal sequences of the final proteins will therefore be ColG: (MIANTNSEKY . . . ) and ColH: (MVQNESKRYT . . . ).

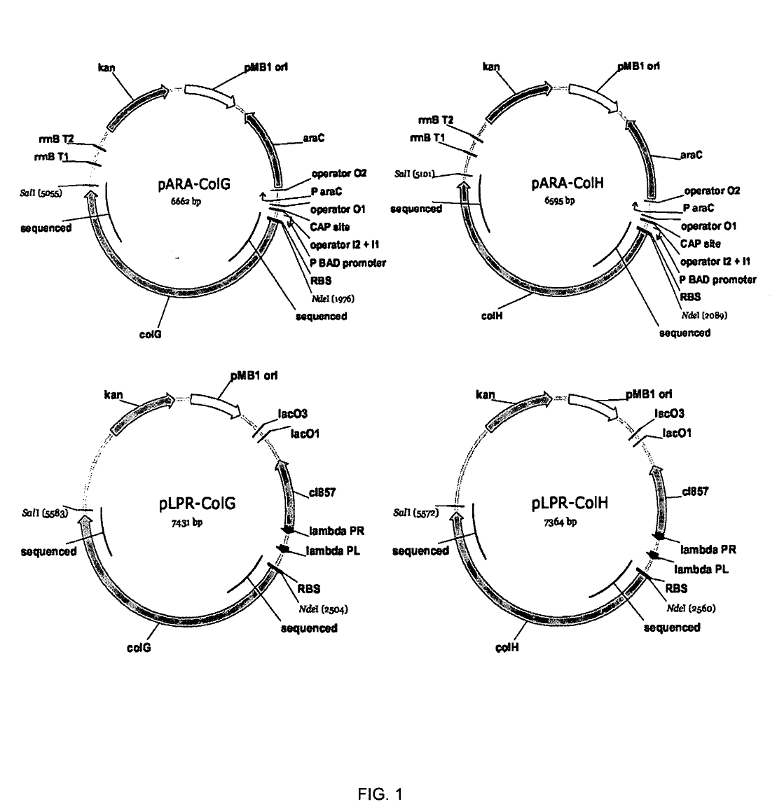

Synthesis and sequencing of the genes was subcontracted to Geneart AG (Regensburg, Germany) and the codon-optimized genes were supplied to Cobra Biologics (Staffordshire, UK) in two plasmids. The colG and colH genes were subcloned from these into two different Cobra expression plasmids in E. coli strain TOP10, with plasmid identity confirmed by restriction analysis and sequencing across the cloning junctions. The four collagenase expressing plasmids (FIG. 1) were tested for stability during repeated subculture, and all four plasmids were found to be structurally and segregationally stable over the course of more than 50 generations in the absence of a selective antibiotic (FIG. 3), demonstrating their suitability for future fermentation production.

Both expression systems enabled high-yield production, with the collagenases representing a significant proportion (up to 40%) of total cellular protein (FIG. 4). The highest yield was from the arabinose system (˜140 mg l⁻¹). These yields were obtained using shake flask cultures with low optical densities—a significant increase in productivity could be achieved by using a fed-batch fermentation strategy, depending on the efficiency of recovery during downstream purification.

The arabinose system produces a mixture of soluble and insoluble protein for both collagenases, and the relative proportions of both forms were not altered significantly by reducing the growth temperature from 37° C. to 30° C. (FIGS. 7 and 8). The lambda system produces insoluble protein due to its requirement for induction at 42° .

With ColG expression there is a lower molecular mass protein (˜80 kDa) that is detected in the western blot. This could be a degradation product, or the result of anomalous initiation or termination of transcription or translation. In the arabinose system the smaller product increases in concentration following induction, suggesting that it is derived from colG. In the lambda system it is already present pre-induction and does not increase significantly in concentration post-induction (FIG. 5). The solubility of this smaller protein was investigated in both systems, which revealed that with arabinose it is present in soluble and insoluble forms in an equivalent ratio to ColG at 30° C. and 37° C. (FIG. 10), but with lambda it shifts from being soluble to insoluble upon induction by a temperature increase to 42° C. (FIG. 12). However, western blots do not give a reliable comparison of protein concentration, and this smaller protein represents a tiny contaminant that is barely (if at all) visible on a polyacrylamide gel, so it is unlikely to be problematic in downstream purification.

In conclusion, two systems have been engineered to produce high levels of recombinant C. histolyticum collagenases ColG and ColH in E. coli from stable, kanamycin-selected plasmids. The arabinose system enables production of soluble or insoluble protein, with the lambda system favoring insoluble production by an inclusion body route. Further investigations are required to determine the yields during fermentation, the purification and activity of soluble collagenases and feasibility of refolding collagenases from inclusion bodies.

Thus, in one embodiment, the invention includes an isolated or purified codon-optimized DNA encoding a collagenase comprising a nucleic acid sequence having at least 95%, preferably at least 96%, 97%, 98%, 99% or 100%, sequence identity to a member of the group selected from SEQ ID 1 and SEQ ID 3, and complements thereof. The isolated or purified codon-optimized DNA consists of SEQ ID 1 and/or 3 and complements thereof.

Variants of ColG and ColH exist wherein several amino acids differ. For example, one variant ColG has 5 amino acid substitutions and one variant ColH has 2 amino acid substitutions. The codon-optimized sequences of the invention can be modified so as to encode such variant collagenase proteins by utilizing substantially the same optimized codons as in SEQ IDs 1 and 3, along with optimized codons encoding the amino acids which differ in those variants. For example, SEQ IDs 1 and 3 may be used as the basis for encoding such variant ColG and ColH having five amino acid differences in ColG from SEQ ID 2 and two amino acid differences from SEQ ID 4, as shown in Tables A and B.

TABLE A
Optimized codons in ColG

| | | Nucleotide | | |
|---|---|---|---|---|
| | | position of | Amino Acid | Amino Acid |
| Codon in | Modified | codon in | (former to | position in |
| SEQ ID 1 | codon(s) | SEQ ID 1. | new) | SEQ ID 2 |
| Ctg | atc or att | 646-648 | L to I | 216 |
| Ggc | cag | 1033-1035 | G to Q | 345 |
| Ggc | cag | 1864-1866 | G to Q | 622 |
| Ggc | cag | 1942-1944 | G to Q | 648 |
| Ctg | atc or att | 2662-2664 | L to I | 888 |

TABLE B
Optimized codons in ColH

| | | Nucleotide | | |
|---|---|---|---|---|
| | | position of | Amino Acid | Amino Acid |
| Codon in | Modified | codon in | (former to | position in |
| SEQ ID 3 | codon(s) | SEQ ID 3. | new) | SEQ ID 4 |
| Ttt | acc or acg | 1189-1191 | F to T | 397 |
| Ctg | atc or att | 1615-1617 | L to I | 539 |

In one embodiment, the DNA for the variant ColG is substantially identical to SEQ ID 1 characterized by one or more of:

“atc” or “att” at nucleotide positions 646-648, encoding I,

“cag” at nucleotide positions 1033-1035, encoding Q,

“cag” at nucleotide positions 1864-1866, encoding Q,

“cag” at nucleotide positions 1942-1944, encoding Q, and

“atc” or “att” at nucleotide positions 2662-2664, encoding I,

and complements thereof.

In another embodiment, the DNA for the variant ColH is substantially identical to SEQ ID 3 characterized by one or more of:

“acc or acg” at nucleotide positions 1189-1191, encoding T, and

“atc” or “att” at nucleotide positions 1615-1617, encoding I,

and complements thereof.

Other variants may similarly be obtained using the teachings herein. In one embodiment, the sequences do not employ codons detrimental to E. coli expression, such as AGG, AGA or CGA (encoding Arg), CTA (encoding leucine), ATA (encoding isoleucine) and CCC (encoding proline). Kane, J. 1995, Current Opinion in Biotechnology, 6: 494-500.

The Genbank ColH sequence has ten extra amino acids on the N-terminus compared to the codon-optimized sequence of SEQ ID 3 (i.e., AVDKNNATAA). However, none of these N-terminal amino acid differences alter the catalytic domains of the collagenases as described in Matsushita et al. 1999.

The invention also relates to a recombinant DNA or DNA molecule obtained, or obtainable by, inserting the isolated or purified codon-optimized DNA as described herein into a vector. Preferably the DNA molecule is operably linked to one or more control sequences. The invention also relates to other nucleic acid molecules corresponding to the DNA molecules described herein, including RNAs and the like.

The recombinant DNA can be inserted, transfected or otherwise transformed into a host cell by known methods, thereby achieving a “transformant” containing the DNA of the invention and capable of expressing collagenase. The host cell is preferably bacterial and is preferably Escherichia coli.

The invention also includes a process for producing collagenase comprising culturing transformant in a medium to form and accumulate in culture a collagenase, and recovering the polypeptide from the culture.

### EXAMPLES

### Example 1

### Materials

The materials and reagents used in the experiments are summarized in Table 1. All restriction enzymes, T4 DNA ligase and CIP (Calf alkaline intestinal phosphatase) were supplied by NEB (New England Biolabs, Ipswich, Mass.). TOP10 cells were obtained from Invitrogen.

TABLE 1
Materials

| | |
|---|---|
| Growth Media | LB medium (pH 7.5 with sodium hydroxide): |
| | 10 g/L Phytone peptone (BD, cat. no. 292450); |
| | 5 g/L yeast extract (BD, cat. no. 212730); |
| | 5 g/L sodium chloride |
| | SOC medium (pH 7 with 5M NaOH): |
| | 10 g/L Phytone peptone (BD, cat. no. 292450); |
| | 5 g/L yeast extract (BD, cat. no. 212730); |
| | 0.5 g/L sodium chloride (VWR BDH AnalaR, |
| | cat. no. 10241AP); 2.5 mM KCl; 50 mM MgCl₂; |
| | 20 mM glucose. |
| | (MgCl2 and Glucose were added after autoclaving) |
| | TB medium: |
| | TB powder (Gibco, cat. no. 22711-022): 47.0 g/L; |
| | 4 ml/L glycerol. |
| Strain genotype | Escherichia coli TOP10 (Invitrogen). |
| | F⁻ mcrA Δ(mrr-hsdRMS-mcrBC) Φ80lacZΔM15 |
| | Δlac deoR recA araD139 Δ(ara-leu)7697 galU |
| | galK rpsL (StrR) endA1 nupG |
| Plasmids | The genes (synthesised and fully sequenced codon- |
| 0600847pUC19 | optimised versions of the collagenase genes) were |
| (colG) and | supplied on plasmids 0600847pUC19 and 0600846pGA4 |
| 0600846pGA4 | from Geneart AG (Regensburg, Germany). |
| (colH) | |

### Example 2

### Collagenase Sequences

Once designed the codon-optimized genes were synthesized, fully sequenced and cloned in plasmids 0600847pUC19 (colG) and 0600846pGA4 (colH).

The codon-optimized ColG DNA is represented herein as SEQ ID NO: 1; while the ColH DNA is represented herein as SEQ ID NO: 3. The proteins encoded by codon-optimized ColG and ColH are represented as SEQ ID NO: 2 and SEQ ID NO: 4, respectively. The codon-optimized sequences of the invention can also encode a protein having five amino acid differences from the Genbank database entry for ColG by Matsushita et al. 1999 (accession number D87215) and two amino acid differences from the Genbank database entry for ColH by Yoshihara et al. 1994, (accession number D29981). These differences are summarized in Tables 2 and 3.

TABLE 2
Optimized codons in ColG

| | Nucleotide position of | Amino Acid | Amino Acid position |
|---|---|---|---|
| Codon | codon in SEQ ID 1. | (former to new) | in SEQ ID 2 |
| ctg | 646-648 | L to I | 216 |
| ggc | 1033-1035 | G to Q | 345 |
| ggc | 1864-1866 | G to Q | 622 |
| ggc | 1942-1944 | G to Q | 648 |
| ctg | 2662-2664 | L to I | 888 |

TABLE 3
Optimized codons in ColH

| | Nucleotide position of | Amino Acid | Amino Acid position |
|---|---|---|---|
| Codon | codon in SEQ ID 3. | (former to new) | in SEQ ID 4 |
| ttt | 1189-1191 | F to T | 397 |
| ctg | 1615-1617 | L to I | 539 |

### Example 3

### Plasmid Construction

The two plasmids 0600847pUC 19 and 0600846pGA4 were digested with NdeI, SalI and ScaI to release a 3.1 kb colG and a 3.05 kb colH fragment. The ScaI digest was used to enable a better agarose gel separation of the vector backbone from the collagenase gene fragments. The expression vectors pORT-LPR(+) (4.36 kb) and pORT-LBAD (3.6 kb) were digested with NdeI and SalI; the fragments were gel-purified and treated with CIP (calf intestinal phosphatase) to prevent religation. Both colG and colH were ligated to each of the vectors resulting in pARAColG, pARA-ColH, pLPR-ColG and pLPR-ColH. Ligations were performed at 16° C. for 3-4 hours. Electrocompetent TOP 10 cells (100 μl) were then transformed with 2 μl of each ligation reaction. Electroporation was carried out in 2 mm cuvettes at 2.5 kV cm⁻¹, 335: and 15 μF. Cells were then incubated for 1 hour in SOC medium at 37° C. (pARA plasmids) or 30° C. (PLPR plsmids) in a shaking incubator (200 rpm). They were then plated onto LB-agar plates (containing kanamycin at 50 μg ml⁻¹) and incubated overnight at 37° C. (pARA plasmids) or 30° C. (pLPR plasmids). Miniprep DNA was generated using a standard kit (Qiagen) and clones were analyzed using restriction enzyme digests and agarose gel electrophoresis to identify correct clones.

### Example 4

### Expression Plasmid Analysis

As expression of collagenase genes colG and colH in E. coli could be toxic to the host, two tightly regulated protein expression systems were used: the arabinose and the lambda promoter-repressors. The arabinose system relies on regulation of the P_{BAD} promoter by the AraC repressor protein; upon addition of arabinose to the growth medium, AraC induces transcription from P_{BAD}. The lambda system makes use of a thermo-labile repressor protein encoded by the cI857 gene to control transcription from the strong tandem P_{L}-P_{R} promoters. Collagenase expression was evaluated in shake flasks, using SDS-PAGE and western blotting techniques on whole cell protein preparations and samples partitioned into soluble and insoluble fractions.

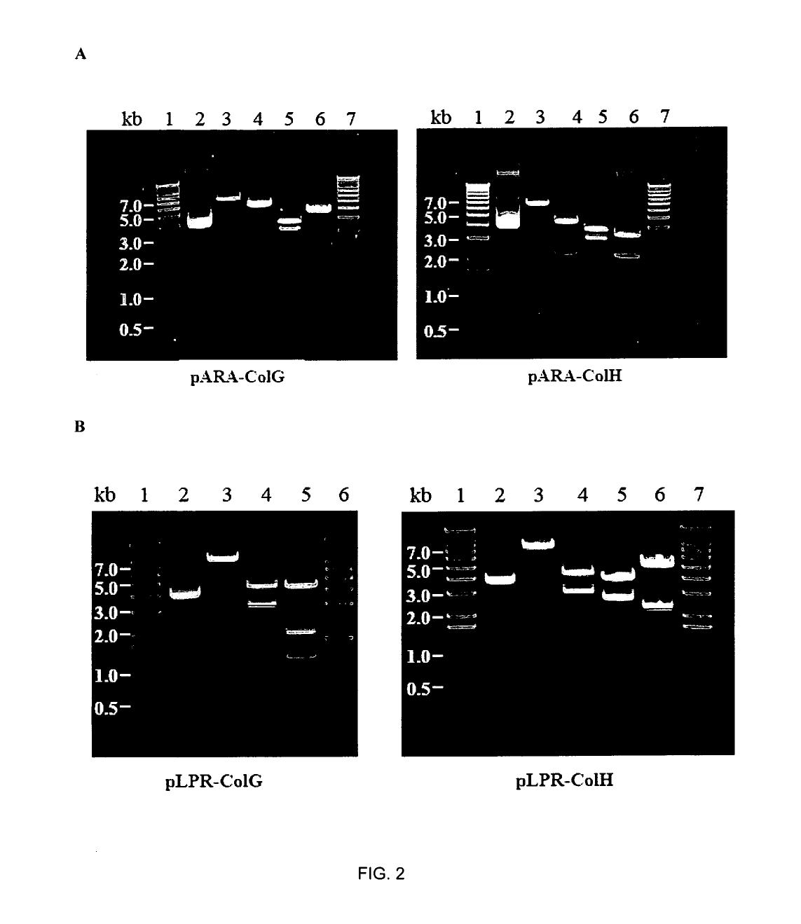

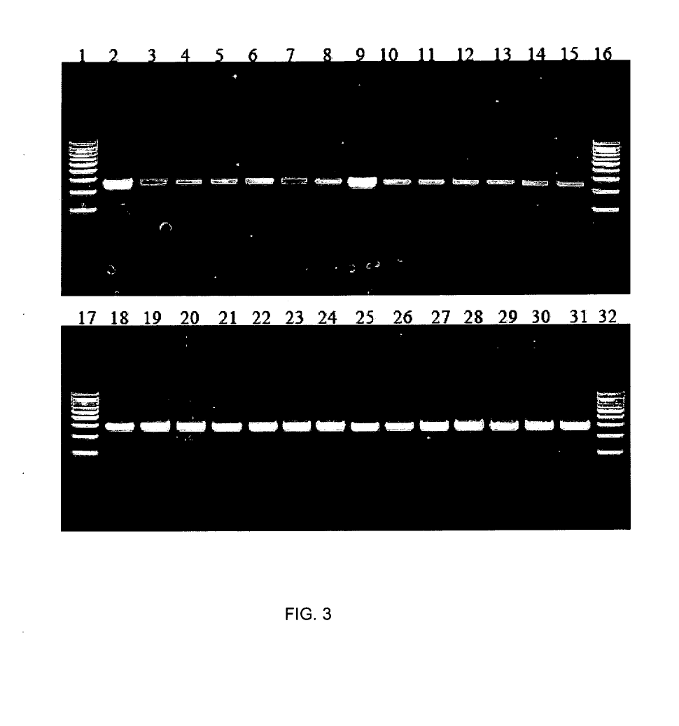

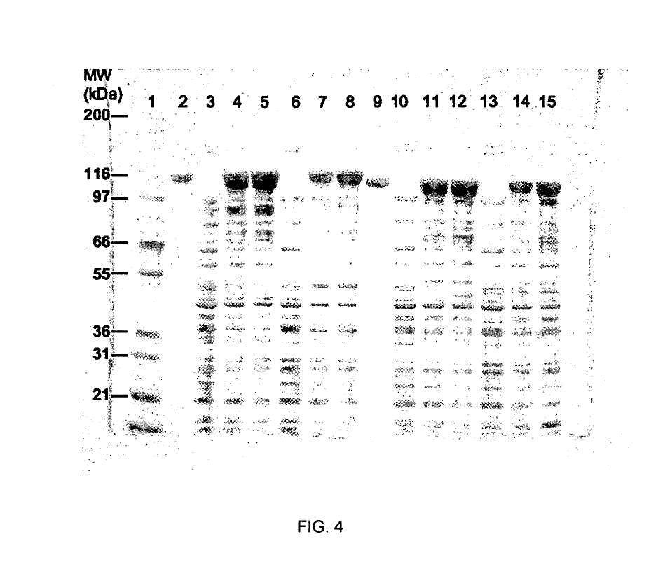

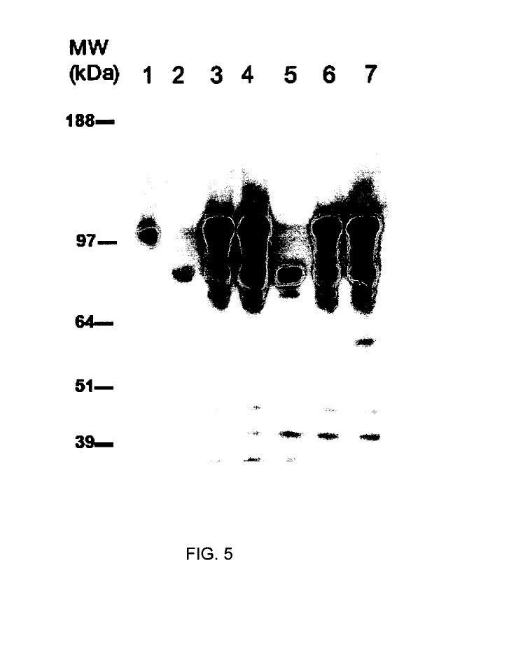

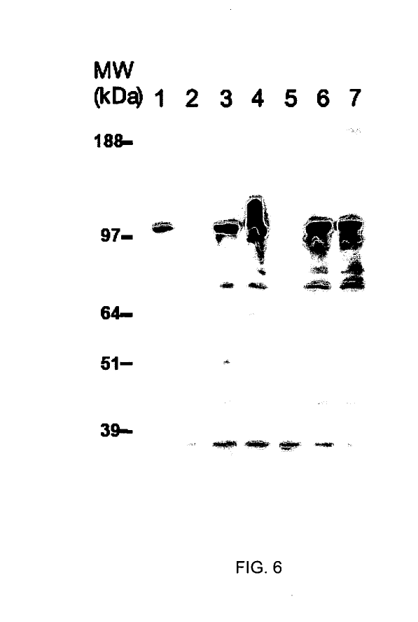

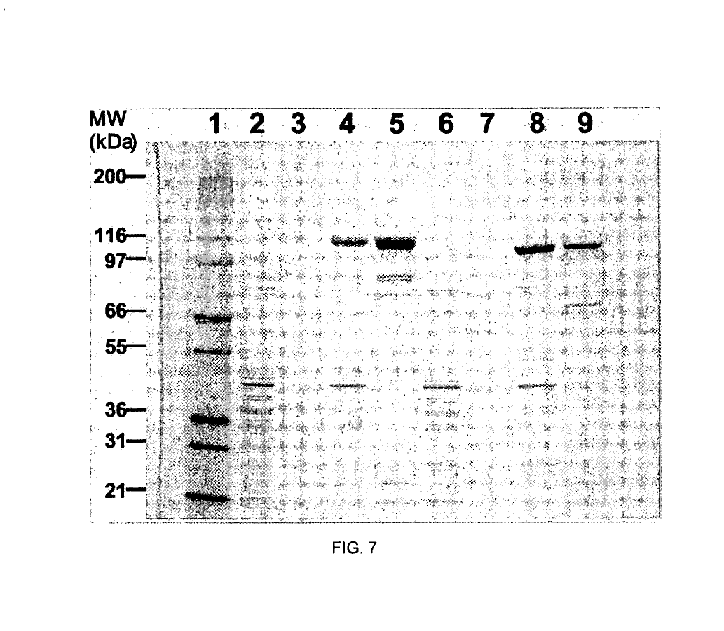

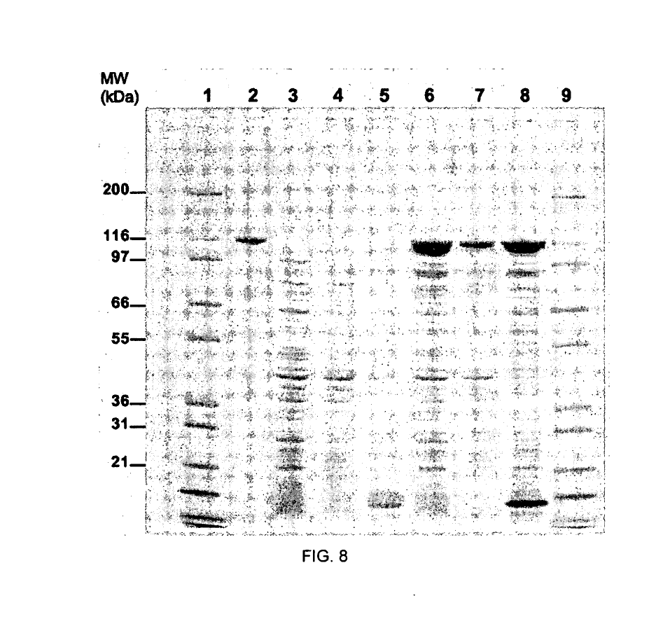

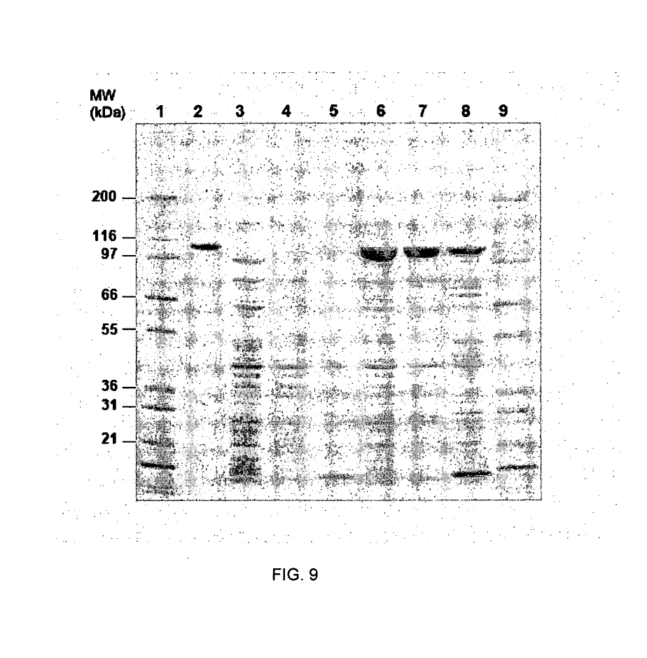

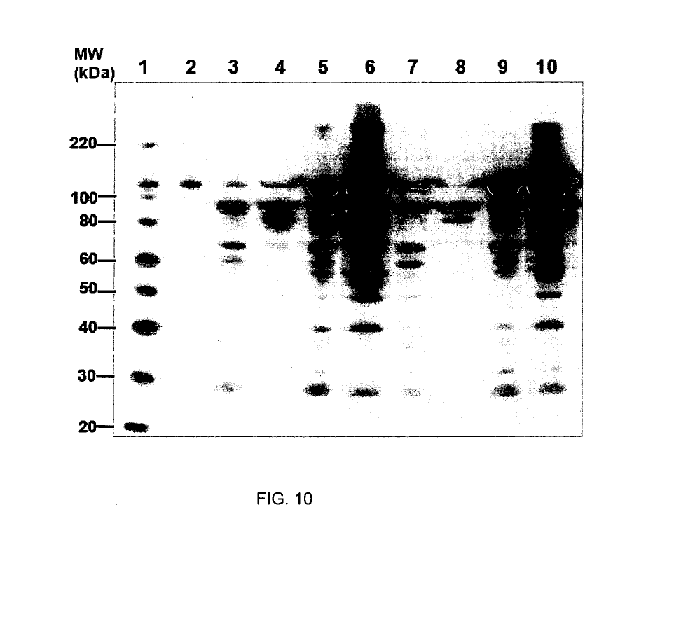

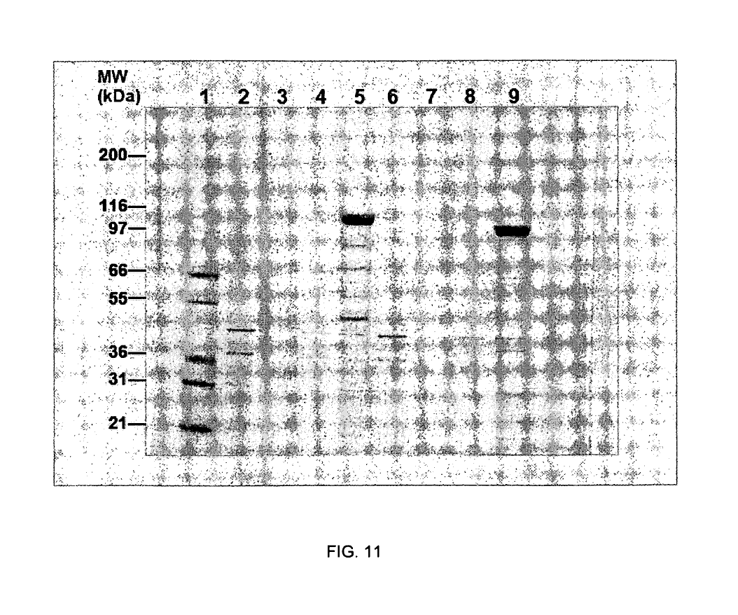

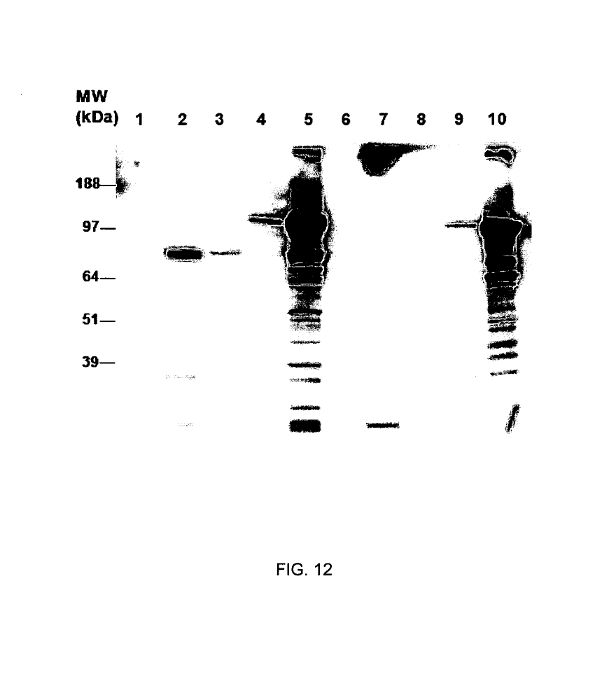

The NdeI-SalI fragments of plasmids 0600847pUC 19 and 0600846pGA4 (containing colG and colH respectively) were cloned into Cobra expression plasmids pORT-LPR and pORT-LBAD cut with the same enzymes, generating pARA-ColG, pARA-ColH, pLPR-ColG and pLPR-ColH as described above. These were transformed into E. coli TOP 10 and plasmid minipreps were performed on selected clones to extract DNA for restriction analysis and sequencing of the cloning junctions. The gel is shown in FIG. 2. These operations confirmed that the correct plasmids had been generated. All samples were run on 0.7% agarose gel in TAE. The sizes of fragments are indicated in brackets. The size marker used was 1 kb Plus DNA ladder (Invitrogen).

### Example 5

### Plasmid Stability

The four TOP10 strains containing the plasmids described above were serially subcultured for more that 50 generations in LB broth without kanamycin, and showed no sign of structural or segregational instability. Plasmids pARA-ColG, pARA-ColH, pLPR-ColG and pLPR-ColH were analyzed on a 0.7% agarose gel in TAE buffer. Approximately 0.4 ug of reference DNA was loaded for each plasmid (lanes 2, 9, 18 and 25). The gel is shown in FIG. 3.

### Example 6

### SDS-PAGE Expression Analysis

To prepare samples for SDS-PAGE analysis, the frozen E. coli cell aliquot equivalent to A₆₀₀=1.0 was resuspended in 125 μl cell-resuspension buffer (50 mM Tris HCl at pH 8, 10 mM MgCl₂) and 1.0 μl Benzonase (Merck, 1.01695.0001) was added. Samples were incubated on ice for 2 hours. For SDS-PAGE loading, 7 μl of the 126 μl sample were mixed with 2 μl 4× sample buffer (NP0007) and 1 μl 10× reducing agent (NP0004). Samples were vortexed briefly and heated for 5 minutes at 95° C., followed by centrifugation at 13000 rpm for 1 minute. Samples were loaded onto NuPAGE Novex 4-12% gradient Bis-Tris gels (Invitrogen, NP0323) in MOPS buffer system along with Mark12 protein marker (Invitrogen, LC5677). Gel electrophoresis was performed at 200V until the blue band entered the lower buffer system.

The gel was fixed in 40% ethanol plus 10% acetic acid for 15 minutes. Staining solution was 25% ethanol and 8% acetic acid containing 0.2% Brilliant Blue ‘R’ (Sigma, B-0 149); this solution was filtered through Whatmann paper 1 (cat. no. 1001 240). Stained gels were de-stained with 25% ethanol and 8% acetic acid. Gels were photographed using an AlphaInnotech imaging system.

Protein expression studies were conducted in Terrific Broth (TB) medium in shake flask cultures, induced at time to by adding 0.2% arabinose or a temperature shift from 30° C. to 42° C. Whole cell (TOP 10 cells) protein extracts were loaded on a 4-12% gradient gel. Samples were taken at the indicated time points (hours post-induction). The molecular weight marker was Mark12 (Invitrogen). Culture volumes containing a number of cells equivalent to an optical density of A₆₀₀=1.0 in 1.0 ml were harvested at the indicated time points, thus normalized for total cellular protein and analyzed by SDS-PAGE. FIG. 4 shows that all four plasmids expressed collagenase at the expected molecular mass as indicated by the reference proteins. There is very little pre-induction expression detected, and the recombinant collagenases represent a significant proportion of total cell protein.

For the arabinose system, protein yield is constant following induction. For the lambda system the protein yield at 3 hours after induction is greater than at 1 hour, even though there was a reduction in culture density after induction (not shown). The amount of collagenase expressed from the arabinose system is approximately 2-fold higher than the lambda system. Based on the gel shown in FIG. 4, the productivity for the 2-hour sample from the arabinose system (lane 4) is estimated to be at least 140 mg l¹ (volumetric yield).

Single colonies were inoculated into 5 ml LB broth containing kanamycin (50 μg ml⁻¹) and incubating over night at the appropriate temperature. All arabinose cultures were incubated at 37° C. and lambda cultures at 30° C. unless otherwise stated. The following day, cultures were diluted to an optical density of A₆₀₀=0. 1 in 50 ml Terrific Broth (TB) medium containing 50 μg ml⁻¹ kanamycin in Erlenmeyer flasks and grown in a shaking incubator (200 rpm). When the cultures reached approximately A₆₀₀=1.0, they were either induced by adding 0.2% arabinose (ARA cultures) or by increasing the temperature from 30° C. to 42° C. (LPR cultures). Samples containing a number of cells equivalent to 1.0 ml of a culture with A₆₀₀=1.0 were then taken at the time of induction (t₀) or at hourly time points. The samples were frozen and later analyzed by SDS-PAGE and Coomassie staining.

### Example 7

### Protein Partitioning

Protein partitioning into soluble and insoluble fractions was examined by separating these fraction from cell pellets using BugBuster (Novagen, cat. no. 70584, following the manufacturer's instructions). A cell sample equivalent to A₆₀₀=0.1 was resuspended in 150 μl BugBuster containing 1 μl Benzonase (250 units μl⁻¹) and incubated on a shaking platform for 10-20 minutes. Cells were centrifuged at 13000 rpm for 20 minutes at 4° C. The supernatant, containing the soluble fraction, was transferred to a fresh Eppendorf tube. The pellet was then resuspended in BugBuster containing 200 μg ml⁻¹ lysozyme, the sample was vortexed and incubated for 5 minutes at room temperature. Next, 6 volumes (900 μl) of a 10-fold dilution of BugBuster was added and the samples vortexed for 1 minute. Samples were centrifugated at 13000 rpm for 15 minutes at 4° C. The supernatant was discarded and the pellet washed with 75 μl 10-fold diluted BugBuster. Samples were centrifuged 15 min 13000 rpm at 4° C. The washing step was then repeated twice. Inclusion bodies were resuspended after final centrifugation in 150 μl TE buffer.

The soluble and insoluble fractions for each plasmid at each time-point were subjected to SDS-PAGE, with a three-fold volumetric excess of the insoluble fraction to compensate for its lower protein concentration (3.3 μl soluble and 10 μl insoluble fractions were loaded for SDS-PAGE analysis).

### Example 8

### Western Blot Materials and Methods

Materials used to perform Western blot analysis are summarized in Table 5. The method was as follows: Following polyacrylamide gel electrophoresis, proteins were blotted onto nitrocellulose at 50 mA for approximately 80 minutes, the membrane was left to dry for 2 minutes on Whatmann paper and blocked with 1×TBS-0.1% T-5% milk for 1 hour at room temperature on a shaking platform. The membrane was incubated with the primary antibody as indicated above for 1 hour at room temperature on a shaking platform and then washed with 1×10 ml 1×TBS-0.1% T which was immediately discarded, then with 1×10 ml 1×TBS-0.1% T for 15 minutes, then with 1×10 ml 1×TBS-0.1% T twice for 5 minutes. The blots were then incubated with the secondary antibody for 30 minutes at room temperature on the shaking platform and washed with 1×10 ml 1×TBS-0.1% T which was then immediately discarded, then with 1×10 ml 1×TBS-0.1% T for 10 minutes, then with 1×10 ml 1×TBS-0.1% Tween for 5 minutes, then with 1×10 ml 1×TBS-0.2% T for 5 minutes, then with 1×10 ml 1×TBS which was immediately discarded and finally with 1×10 ml 1×TBS for 5 minutes.

The membrane was incubated for 5 minutes at room temperature with 4 ml of chemiluminescence mix solution and finally placed between overhead projector acetates. Chemiluminescence was detected with the AlphaInnotech imaging system.

TABLE 5
Western blot materials

| | |
|---|---|
| Native reference proteins: | ColG: 0.55 μg μl⁻¹ and ColH: 0.54 μg μl⁻¹ |
| | (Auxilium) |
| Protein marker: | Prestained SeeBlue Plus2 (LC5925), |
| | Mark12 (Invitrogen) |
| Blotting device: | Trans-Blot SD Semidry-Transfer Cell |
| Blotting membrane: | Gelman Science Nitrocellulose (cat. no. |
| | 66489) |
| Blotting buffer: | 20% methanol, 0.037% SDS, 48 mM Tris |
| | base, 39 mM glycine (0.2 μm sterile- |
| | filtered) |
| 10x TBS: | 100 mM Tris-HCl, pH 8 |
| (Tris-Buffered-Saline) | 1.5M NaCl (10xTBS was made up with |
| | sterile solutions) |
| 1x TBS: | 1:10 diluted 10xTBS |
| 1x TBS-0.1% T: | 1xTBS with 0.1% (v/v) Tween20 |
| 1x TBS-0.2% T: | 1xTBS with 0.2% (v/v) Tween20 |
| 1x TBS-0.1% T-5% milk: | 1xTBS with 0.1% (v/v) Tween20 and 5% |
| | (w/v low-fat milk powder (Sainsbury)) |
| 1x TBS-0.1% T-0.5% milk: | 1xTBS with 0.1% (v/v) Tween20 and 0.5% |
| | (w/v) low-fat milk powder; made up by |
| | diluting the 5% milk buffer 1:10 with |
| | 1xTBS-0.1% T |
| Primary antibody: | Rabbit affinity purified anti-ColG (Aux I) |
| | and rabbit affinity purified anti-ColH |
| | (AuxII) (Auxilium); both diluted 1:20000 |
| | with 1xTBS-0.1% T-5% milk |
| Secondary Antibody: | Dako anti-rabbit IgG-HRP from goat (cat. |
| | no. P0448) at 1:2,000 diluted with 1xTBS- |
| | 0.1% T-0.5% milk. |
| Chemiluminescence: | Sigma C9107/C9232 solutions mixed in a |
| | 1:2 volume ratio |
| | (approximately 4 ml total volume used for |
| | each blot) |

### Example 9

### Plasmid Stability Study

The plasmids were tested for structural and segregational stability by serial subculture in 5 ml of LB broth (in 30 ml tubes). On ‘day 0’ strains were inoculated from single colonies into LB broth with kanamycin and incubated overnight at 30° C. The optical density was determined by reading the absorbance at 600 nm. A volume containing the number of cells equivalent to an optical density of A₆₀₀=2.0 in 1.0 ml was extracted and centrifuged, the supernatant removed and the cell pellet frozen. The cultures for ‘day 1’ (no antibiotic) were inoculated to a starting optical density of A₆₀₀=0.001 and incubated as before. Subculture at 30° C. in the absence of antibiotic was continued until ‘day 5’, by which time each strain had grown for over 50 generations. Plasmid DNA was extracted from the cell pellets in a total volume of 50 μl Tris-HCl and 9 μl was used for agarose gel electrophoresis. Generation numbers were calculated using the equation: number of generations=1n(ΔA₆₀₀)/1n2.

### Example 10

### Western Blot Analysis of ColG Expression

Cells were grown and the western blots performed as described in the Material and Methods section. The western blot of ColG is shown in FIG. 5.

The western blot illustrates that the levels of pre-induction collagenase expression from both promoters are very low (lanes 2 and 5). The molecular mass of ColG expressed from both plasmids is the same size as the reference (lane 1). Additionally, a lower molecular mass band can be seen in the lambda system in the uninduced (lane 5) and induced states (lane 6, 7). This protein has a molecular mass of approximately 80 kDa and its concentration does not increase post-induction. For the arabinose system the concentration of this band is much lower for the uninduced samples (lane 2) but increases after induction (lane 3, 4) to levels similar to the lambda system.

This lower molecular mass protein may be the result of protein degradation, expression resulting from an internal promoter-like sequence within the ColG cistron or premature termination of transcription or translation. However, degradation would appear less likely due to the higher concentration of this smaller protein relative to ColG in the uninduced samples.

### Example 11

### Western Blot Analysis of ColH Expression

Cells were grown and the western blots performed as described in the Material and Methods section. The western blot of ColH is shown in FIG. 6.

The molecular mass of ColH expressed from both plasmids in FIG. 6 is equal to the size of the reference ColH (lane 1). The apparently larger ColH band in lane 4 is likely to be an artifact of gel electrophoresis. For both expression systems, no ColH can be detected pre-induction under these conditions (lane 2 and 5). Relatively low amounts of lower molecular mass protein are produced, presumably due to a small amount of collagenase degradation.

### Example 12

### Solubility of ColG and ColH from the Arabinose System at 37° C.

Protein preparations from cultures grown at 37° C. using the arabinose system were partitioned into soluble and insoluble fractions and analyzed by SDS-PAGE. Volumes were loaded to correct for lower protein content of the insoluble fraction were 3.3 μl soluble and 10 μl insoluble fraction. Samples for the time-points t0 and t2 of the expression experiment were analyzed.

ColG and ColH were detected in both fractions and the data are shown in FIG. 7. Neither of the collagenases was detected at induction time point, t0. Higher proportions of ColG were found in the insoluble fraction compared to ColH (compare lanes 5 and 9). To enable comparison of total cell protein, a three-fold volumetric excess of the soluble sample over the insoluble sample was loaded. It is estimated that 70-80% of ColH is found in the soluble fraction whereby for ColG this fraction is approximately 30-40%.

### Example 13

### Solubility of ColG and ColH in the Arabinose System at 30° C.

As the collagenases produced with the arabinose system in E. coli are a mixture of soluble and insoluble proteins at 37° C., a further expression study was conducted to investigate if reducing the growth temperature to 30° C. would increase the proportion of soluble protein. The results in FIGS. 8 and 9 indicate that the lower growth temperature did not significantly alter the distribution between the soluble and insoluble cellular fractions for ColG and ColH (compare lanes 7 and 8). Samples of total cell protein were also loaded (lanes 3 and 6), indicating that the lower growth temperature did not significantly reduce collagenase yield.

### Example 14

### Solubility of the Lower Molecular Weight ColG Product in the Arabinose System

It was decided to investigate if the lower molecular mass contaminant observed in western blots for ColG expression (FIG. 5) partitioned to the soluble or insoluble fraction, as it may influence the choice of downstream purification strategy. Partitioned samples from cultures grown at both 30° C. and 37° C. were analyzed by western blot (FIG. 10). This revealed that the lower molecular mass ColG-derivative is present in both the soluble and insoluble fractions, in approximately the same ratio as ColG. However, as this smaller protein is only detectable by western blot, it represents a very minor contaminant.

### Example 15

### Solubility of ColG and ColH in the Lambda System

Partitioning studies were carried out on cell extracts from the lambda expression studies and samples at time-points t0 and t3were analyzed by SDS-PAGE. This revealed that ColG and ColH are mainly found in the insoluble fraction, presumably as inclusion bodies, and based on the gel loading it can be estimated that this fraction represents about 90-95% of the total recombinant collagenase protein. The greater plasmid copy number and 42 ° C. induction temperature is known to favor the production of inclusion bodies. For the soluble fractions 3.3 μl and for the insoluble fractions 10 μl were loaded to correct for lower protein content of the insoluble fraction.

### Example 16

### Solubility of the Lower Molecular Mass ColG Band in the Lambda System

Previous western blot analysis of the lambda expression system (FIG. 5) revealed a lower molecular mass protein that hybridized to the ColG antibody, and that was surprisingly produced at equivalent concentrations pre- and post-induction. Studies were therefore undertaken to determine whether this is present in the soluble or insoluble fraction after induction. ColH was investigated simultaneously. Cultures of TOP 10 containing pLPR-ColG and pLPR-ColH were grown at 30° C. as described previously and induced by a temperature shift to 42° C., and samples were taken at t0and t3. Soluble and insoluble fractions were then prepared and analyzed by western blotting. The data are shown in FIG. 12.

As observed before (see FIG. 5), the lower molecular mass band of ColG is present at t0(uninduced state) and is predominantly present in the soluble fraction (compare lanes 2 and 3). After temperature induction this lower molecular mass band shifts from the soluble fraction to the insoluble fraction (compare lanes 4 and 5). As before, no equivalent lower molecular mass band can be seen for the ColH protein samples.

While this invention has been particularly shown and described with references to preferred embodiments thereof, it will be understood by those skilled in the art that various changes in form and details may be made therein without departing from the scope of the invention encompassed by the appended claims.

1. An isolated or purified codon-optimized DNA encoding a collagenase comprising a nucleic acid sequence having at least 95% sequence identity to a member of the group selected from SEQ ID 1 and SEQ ID 3, and complements thereof.

2. The isolated or purified codon-optimized DNA of claim 1 having at least two optimized codons wherein one of the optimized codons is ctg.

3. The isolated or purified codon-optimized DNA of claim 2 wherein the optimized codons of SEQ ID 1 comprise one or more:
“atc” or “att” at nucleotide positions 646-648,
“gcg” at nucleotide positions 1033-1035,
“gcg” at nucleotide positions 1864-1866,
“gcg” at nucleotide positions 1942-1944, and
“atc” or “att” at nucleotide positions 2662-2664 and complements thereof.

4. The isolated or purified codon-optimized DNA of claim 2 wherein the optimized codons of SEQ ID 3 comprise:
“acc” at nucleotide positions 1189-1191, and/or
“atc” or “att” at nucleotide positions 1615-1617 and complements thereof.

5. The isolated or purified codon-optimized DNA of claim 1 which consists of SEQ ID 1.

6. The isolated or purified codon-optimized DNA of claim 1 which consists of SEQ ID 3.

7. A recombinant DNA obtained by inserting the isolated or purified codon-optimized DNA according to claim 1 into a vector.

8. The recombinant DNA of claim 7, wherein said isolated or purified codon-optimized DNA is operably linked to one or more control sequences recognized by a host cell transformed with the vector.

9. A transformant obtained by introducing the recombinant DNA according to claim 7 into a host cell.

10. The transformant of claim 9 wherein the host cell is a bacterium.

11. The transformant of claim 10 wherein the bacterium is E. coli.

12. A process for producing a collagenase, which comprises
a. culturing the transformant according to claim 11 in a medium to form and accumulate in culture a collagenase and
recovering the collageanse from the culture.
